(19) United States
(12) Patent Application Publication
(10) Pub. No.: US 2026/0151104 A1
(43) Pub. Date: Jun. 4, 2026

(54) ULTRASONIC PROBE AND ULTRASONIC ENDOSCOPE

(71) Applicant: OLYMPUS MEDICAL SYSTEMS CORP., Tokyo (JP)

(72) Inventor: Satoshi YOSHIDA, Tokyo (JP)

(73) Assignee: OLYMPUS MEDICAL SYSTEMS CORP., Tokyo (JP)

(21) Appl. No.: 19/460,575

(22) Filed: Jan. 27, 2026

Related U.S. Application Data
(63) Continuation-in-part of application No. PCT/JP2023/032426, filed on Sep. 5, 2023.

(51) Int. Cl. A61B 8/12; A61B 8/00

(52) U.S. Cl. CPC A61B 8/12; A61B 8/4488

(57) ABSTRACT

An ultrasonic probe includes: a plurality of ultrasonic transducers that are arranged along an arrangement direction from a first side toward a second side; a first flexible printed circuit including a first wiring group that extends from the plurality of ultrasonic transducers toward the first side and electrically connects the plurality of ultrasonic transducers and a plurality of signal lines configured to transmit and receive ultrasonic signals to and from the plurality of ultrasonic transducers; and a second wiring group that extends from the plurality of signal lines toward the second side; a second flexible printed circuit including a third wiring group that extends from the plurality of ultrasonic transducers toward the second side; and a connection portion that electrically connects the second wiring group and the third wiring group.

### CROSS REFERENCES TO RELATED APPLICATIONS

This application is a continuation-in-part of International Application No. PCT/JP2023/032426, filed on Sep. 5, 2023, the entire contents of which are incorporated herein by reference.

### BACKGROUND

### 1. Technical Field

The present disclosure is related to an ultrasonic probe and an ultrasonic endoscope.

### 2. Related Art

In the related art, an ultrasonic endoscope is used to observe an organ of a subject, such as a patient. The ultrasonic endoscope includes a plurality of ultrasonic transducers that are arranged along a predetermined arrangement direction on a distal end of an insertion portion that is inserted into the subject, and a signal cable including a plurality of signal lines is connected to a proximal end side of the insertion portion. The ultrasonic transducers and the signal lines are electrically connected to one another via a relay board that is arranged on back surfaces of the ultrasonic transducers, in a distal end portion of the ultrasonic endoscope.

Meanwhile, it is demanded to reduce a size of the distal end portion of the ultrasonic endoscope to make insertion into the subject easy. International Publication No. WO 2020/079855 discloses a technology for reducing a size of a distal end portion of an ultrasonic endoscope by electrically connecting ultrasonic transducers and signal lines via a flexible printed circuit. In this technology, wires are drawn from the ultrasonic transducers to both of a distal end side and a proximal end side to prevent increase in density of wiring patterns. If the density of the wiring patterns increases, it is needed to reduce widths of the wiring patterns, so that it becomes difficult to increase image quality due to a loss caused by increase in an electrical resistance value, which is not preferable.

### SUMMARY

In some embodiments, an ultrasonic probe includes: a plurality of ultrasonic transducers that are arranged along an arrangement direction from a first side toward a second side; a first flexible printed circuit including a first wiring group that extends from the plurality of ultrasonic transducers toward the first side and electrically connects the plurality of ultrasonic transducers and a plurality of signal lines configured to transmit and receive ultrasonic signals to and from the plurality of ultrasonic transducers; and a second wiring group that extends from the plurality of signal lines toward the second side; a second flexible printed circuit including a third wiring group that extends from the plurality of ultrasonic transducers toward the second side; and a connection portion that electrically connects the second wiring group and the third wiring group.

In some embodiments, an ultrasonic probe includes: a plurality of ultrasonic transducers that are arranged along an arrangement direction from a first side toward a second side; a signal cable including a plurality of signal lines, each signal line being electrically connected to a corresponding one of the plurality of ultrasonic transducers to transmit and receive an ultrasonic signal to and from the corresponding one of the plurality of ultrasonic transducers, each signal line including an exposed portion which is located at a distal end of the single cable and at which a conducting wire for transmitting and receiving the ultrasonic signal is exposed, and a covered portion which is located on a proximal end side of the exposed portion and at which the conducting wire is covered by an outer skin; a flexible printed circuit including a first wiring group that extends from the plurality of ultrasonic transducers toward the first side and electrically connects the plurality of ultrasonic transducers and the plurality of signal lines; and a third wiring group that extends from the plurality of ultrasonic transducers toward the second side and electrically connects the plurality of ultrasonic transducers and the plurality of signal lines; a housing that houses the plurality of ultrasonic transducers, the flexible printed circuit, and exposed portions of the plurality of signal lines; and a bonding portion that bonds a portion of the flexible printed circuit at the first side and a portion of the flexible printed circuit at the second side.

In some embodiments, an ultrasonic endoscope includes: an ultrasonic probe that includes a plurality of ultrasonic transducers that are arranged along an arrangement direction from a first side toward a second side; a first flexible printed circuit including a first wiring group that extends from the plurality of ultrasonic transducers toward the first side and electrically connects the plurality of ultrasonic transducers and a plurality of signal lines configured to transmit and receive ultrasonic signals to and from the plurality of ultrasonic transducers; and a second wiring group that extends from the plurality of signal lines toward the second side; a second flexible printed circuit including a third wiring group that extends from the plurality of ultrasonic transducers toward the second side; a connection portion that electrically connects the second wiring group and the third wiring group; and a housing that houses the plurality of ultrasonic transducers; a rigid material that is connected to the first side of the ultrasonic probe; a bending portion that is connected to the rigid material at the first side and that is bendable; a flexible tube that is connected to the bending portion at the first side and that has flexibility; an insertion portion configured to be inserted into a subject; and an operating portion that is arranged on the first side relative to the insertion portion and is configured to receive operation of bending the bending portion. The connection portion is located on the second side relative to the bending portion.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a configuration of an endoscope system including an ultrasonic probe according to a first embodiment;

FIG. 2 is a cross-sectional view of the ultrasonic probe;

FIG. 3 is a top view of an FPC;

FIG. 4 is a bottom view of the FPC;

FIG. 5 is a partial cross-sectional view of the FPC;

FIG. 6 is an enlarged view of a connection portion in FIG. 2;

FIG. 7 is a flowchart illustrating a method of manufacturing the ultrasonic probe;

FIG. 8 is a partial enlarged view of a connection portion of an ultrasonic probe according to a first modification;

FIG. 9 is a top view of an FPC of an ultrasonic probe according to a second modification;

FIG. 10 is a bottom view of the FPC of the ultrasonic probe according to the second modification;

FIG. 11 is a partial enlarged view of a connection portion of the ultrasonic probe according to the second modification;

FIG. 12 is a partial enlarged view of a connection portion of an ultrasonic probe according to a third modification;

FIG. 13 is a partial enlarged view of an FPC of an ultrasonic probe according to a fourth modification; and

FIG. 14 is a cross-sectional view of an ultrasonic probe according to a second embodiment.

### DETAILED DESCRIPTION

Embodiments of an ultrasonic probe and an ultrasonic endoscope will be described below with reference to the drawings. The present invention is not limited by the embodiments below. The present invention may be generally applied to an ultrasonic probe and an ultrasonic endoscope.

In addition, in the description of the drawings, the same or corresponding components are appropriately denoted by the same reference symbols. Further, it is necessary to note that the drawings are schematic, and dimensional relations among the components, ratios among the components, and the like may be different from the actual ones. The drawings may include portions that have different dimensional relations or ratios.

### First Embodiment

### Overall Configuration of Endoscope System

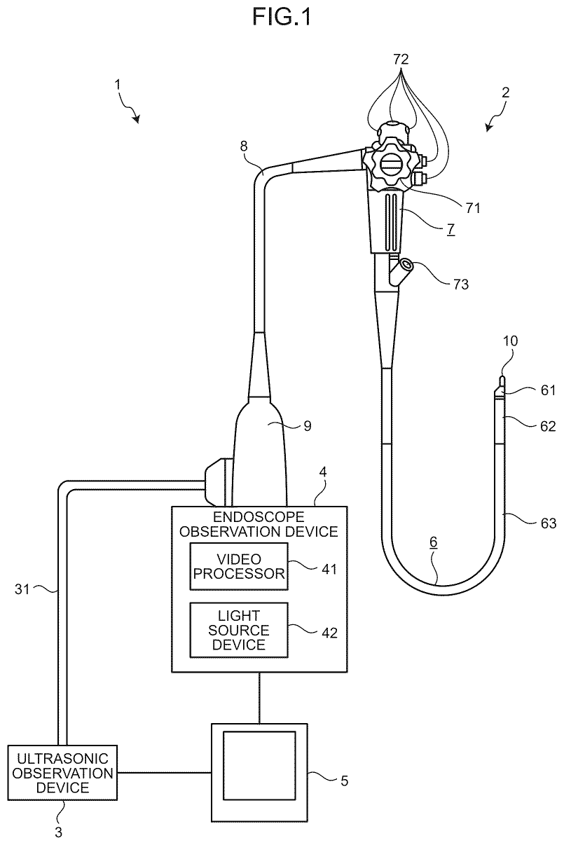

FIG. 1 is a schematic diagram illustrating a configuration of an endoscope system that includes an ultrasonic probe according to a first embodiment. An endoscope system 1 is a system that performs ultrasonic diagnosis and treatment in a subject, such as a human being, by using an ultrasonic endoscope. The endoscope system 1 includes, as illustrated in FIG. 1, an ultrasonic endoscope 2, an ultrasonic observation device 3, an endoscope observation device 4, a display device 5, an insertion portion 6, an operating portion 7, a universal cord 8, and an endoscope connector 9.

In the following, a distal end side of the insertion portion 6 (a distal end side in an insertion direction into the subject) will be described as a “distal end side”, and a proximal end side of the insertion portion 6 (a side away from a distal end of the insertion portion 6) will be described as a “proximal end side”.

The ultrasonic endoscope 2 is partly insertable into the subject, and has a function to transmit ultrasonic pulses (acoustic pulses) to a body wall in the subject, receive ultrasonic echoes reflected by the subject, and output an echo signal, and a function to capture an image in the subject and output an image signal.

The ultrasonic observation device 3 is electrically connected to the ultrasonic endoscope 2 via an ultrasonic cable 31, outputs a pulse signal to the ultrasonic endoscope 2 via the ultrasonic cable 31, and receives the echo signal from the ultrasonic endoscope 2 via the ultrasonic cable 31. Further, the ultrasonic observation device 3 generates an ultrasonic image by performing a predetermined process on the echo signal.

The endoscope connector 9 of the ultrasonic endoscope 2 is removably connected to the endoscope observation device 4. The endoscope observation device 4 includes a video processor 41 and a light source device 42.

The video processor 41 receives input of the image signal from the ultrasonic endoscope 2 via the endoscope connector 9. Further, the video processor 41 generates an endoscopic image by performing a predetermined process on the image signal.

The light source device 42 supplies illumination light for illuminating the inside of the subject to the ultrasonic endoscope 2 via the endoscope connector 9.

The display device 5 is configured by using liquid crystal, organic Electro Luminescence (EL), Cathode Ray Tube (CRT), or a projector, and displays the ultrasonic image that is generated by the ultrasonic observation device 3, the endoscopic image that is generated by the endoscope observation device 4, or the like.

The insertion portion 6 is a portion that is to be inserted into the subject. The insertion portion 6 includes an ultrasonic probe 10 that is arranged on the distal end side, a rigid material 61 that is connected to a proximal end side of the ultrasonic probe 10, a bending portion 62 that is connected to a proximal end side of the rigid material 61 and that is bendable, and a flexible tube 63 that is connected to a proximal end side of the bending portion 62 and that has flexibility.

Meanwhile, in the insertion portion 6, in the operating portion 7, in the universal cord 8, and in the endoscope connector 9, a light guide for transmitting the illumination light supplied from the light source device 42, a signal cable for transmitting the pulse signal and the echo signal, and an image signal cable for transmitting the image signal are pulled around, and a pipeline for circulating fluids is arranged.

The operating portion 7 is a part that is connected to a proximal end side of the insertion portion 6 and receives various kinds of operation from a doctor or the like. The operating portion 7 includes a bending knob 71 for performing bending operation on the bending portion 62 and a plurality of operating members 72 for performing various kinds of operation.

The universal cord 8 is a cord that extends from the operating portion 7 and in which a tube that constitutes a part of the light guide, the signal cable, the image signal cable, and the pipeline is arranged.

The endoscope connector 9 is arranged on an end portion of the universal cord 8. Further, the endoscope connector 9 is connected to the ultrasonic cable 31, and is connected to the video processor 41 and the light source device 42 by being inserted into the endoscope observation device 4.

### Configuration of Ultrasonic Probe

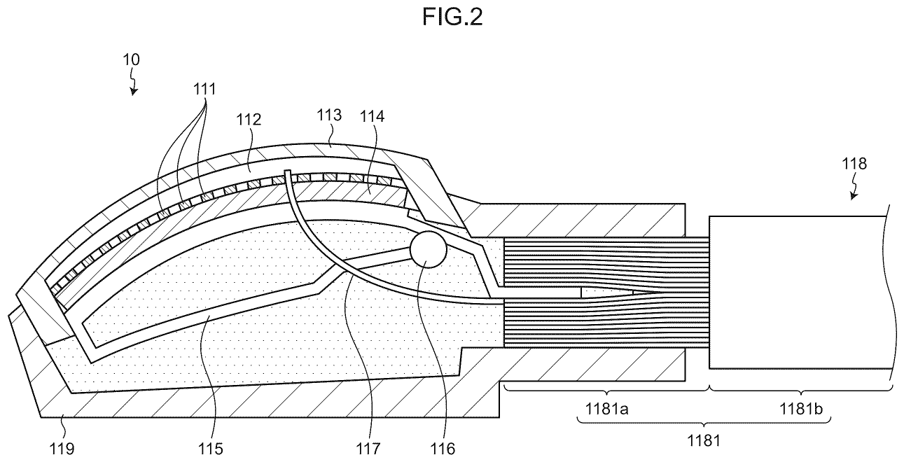

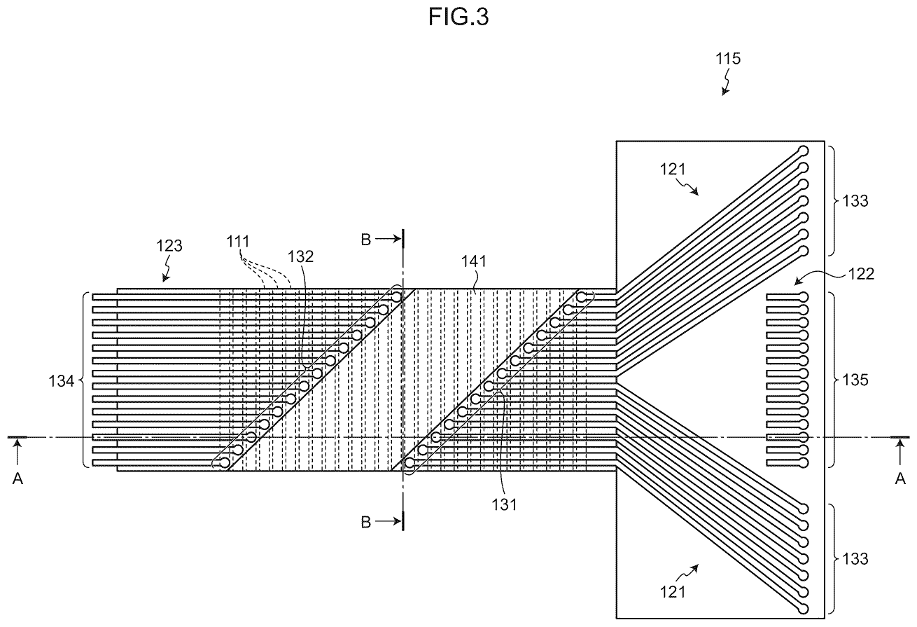

A configuration of the ultrasonic probe 10 will be described below. FIG. 2 is a cross-sectional view of the ultrasonic probe and corresponds to a cross section taken along a line A-A in FIG. 3 (a direction along the line A-A is an arrangement direction of ultrasonic transducers). However, FIG. 2 illustrates a state in which an FPC 115 in FIG. 3 is folded.

The ultrasonic probe 10 is a convex type ultrasonic probe, and has a scanning plane of a cylindrical surface shape that protrudes outward (upward in FIG. 2). The ultrasonic probe 10 includes a plurality of ultrasonic transducers 111, an acoustic matching layer 112, an acoustic lens 113, a back layer 114, the flexible printed circuit (FPC) 115, a connection portion 116, a ground line 117, a signal cable 118, and a housing 119.

Meanwhile, in the following, an example will be described in which a first side corresponds to the proximal end side in a state in which the FPC 115 is expanded as illustrated in FIG. 3, and a second side corresponds to the distal end side in the state in which the FPC 115 is expanded as illustrated in FIG. 3, but embodiments are not limited to this example. Further, a surface of the FPC 115 on the side of the ultrasonic transducers 111 will be described as a “first surface”, and a different surface of the FPC 115 from the first surface will be described as a “second surface”. However, in the following, in the case like, for example, a three-dimensional wiring board in which wiring is performed on three or more surfaces instead of a substrate that extends flatly like the FPC 115, the “second surface” may be a side surface of the first surface.

The plurality of ultrasonic transducers 111 are arranged along the arrangement direction from the proximal end side toward the distal end side. The ultrasonic transducers 111 are located on the distal end side relative to the signal cable 118.

The ultrasonic transducers 111 are formed by using a PMN-PT single crystal, a PMN-PZT single crystal, a PZN-PT single crystal, a PIN-PZN-PT single crystal, or a relaxer material. Meanwhile, the PMN-PT single crystal is an abbreviation of a solid solution of lead magnesium niobate and lead titanate. The PMN-PZT single crystal is an abbreviation of a solid solution of lead magnesium niobate and lead zirconate titanate. The PZN-PT single crystal is an abbreviation of a solid solution of lead zinc niobate and lead titanate. The PIN-PZN-PT single crystal is an abbreviation of a solid solution of lead indium niobate, lead zinc niobate, and lead titanate. The relaxer material is a general term of a ternary system piezoelectric material that is obtained by adding lead-based complex perovskite as a relaxer material to lead zirconate titanate (PZT) for the purpose of increasing a piezoelectric constant or permittivity. The lead-based complex perovskite is represented by Pb(B1, B2)O₃, where B1 is any of magnesium, zinc, indium, and scandium and B2 is any of niobium, tantalum, and tungsten.

The acoustic matching layer 112 is located, with respect to the ultrasonic transducers 111, in a direction in which each of the ultrasonic transducers 111 transmits ultrasonic waves. The acoustic matching layer 112 performs acoustic impedance matching between the ultrasonic transducers 111 and an observation target to efficiently transmit sound (ultrasonic waves) between the ultrasonic transducers 111 and the observation target. The acoustic matching layer 112 may be a single layer or two or more layers. Further, depending on characteristics of the ultrasonic transducers 111 and the observation target, the acoustic matching layer need not always be provided.

The acoustic lens 113 is formed by using silicone, polymethylpentene, epoxy resin, polyetherimide, or the like, has one surface formed in a convex shape or a concave shape to implement a function to focus ultrasonic waves, and emit the ultrasonic waves that have passed through the acoustic matching layer 112 to outside or capture ultrasonic echoes from outside. The acoustic lens 113 may be arranged arbitrarily, and a configuration that does not include the acoustic lens 113 is acceptable.

The back layer 114 is laminated on the ultrasonic transducers 111 on an opposite side of the acoustic matching layer 112, and serves as a dematching layer that reflects at least a part of unneeded ultrasonic waves that are generated by operation of the ultrasonic transducers 111 or a backing member that attenuates unneeded ultrasonic waves that are generated by operation of the ultrasonic transducers 111. Meanwhile, depending on characteristics of the ultrasonic transducers 111 and the observation target, the back layer 114 need not always be provided.

The FPC 115 electrically connects the plurality of ultrasonic transducers 111 and a plurality of signal lines 1181 of the signal cable 118.

The connection portion 116 fix the second surface of the FPC 115 at the distal end side and the second surface of the FPC 115 at the proximal end side. It is preferable to arrange the connection portion 116 at a position on the proximal end side relative to the ultrasonic transducers 111. It is preferable to arrange the connection portion 116 at a position on the distal end side relative to the bending portion 62. This is to prevent stress due to bending from being applied to the connection portion 116.

The ground line 117 electrically connects the ultrasonic transducers 111 and a ground line of the signal cable 118.

The signal cable 118 includes the plurality of signal lines 1181. Each signal line 1181 is electrically connected to a corresponding one of the ultrasonic transducers 111 to transmit and receive an ultrasonic signal to and from the corresponding one of the ultrasonic transducers 111. Each of the signal lines 1181 is a coaxial line that includes a center conductor for transmitting a pulse signal or an echo signal, and a ground line that is arranged around the center conductor in an insulating manner. The signal cable 118 includes exposed portions 1181a which are located at a distal end of the signal cable 118 and at which the signal lines 1181 are exposed, and a covered portion 1181b which is located on a proximal end side of the exposed portions 1181a and at which the signal lines 1181 is covered by an outer skin. The exposed portions 1181a are portions at which a wrapping tape of the signal cable 118 and the outer skin of the signal cable 118 are removed and the signal lines 1181 are exposed and untwisted. The center conductor of each of the coaxial lines is exposed at a distal end of each of the exposed portions 1181a (not illustrated), so that each of the signal lines 1181 is connected to each of connecting terminals (to be described later).

The housing 119 houses the plurality of ultrasonic transducers 111, the FPC 115, and the exposed portions 1181a of the plurality of signal lines 1181.

### Configuration of FPC

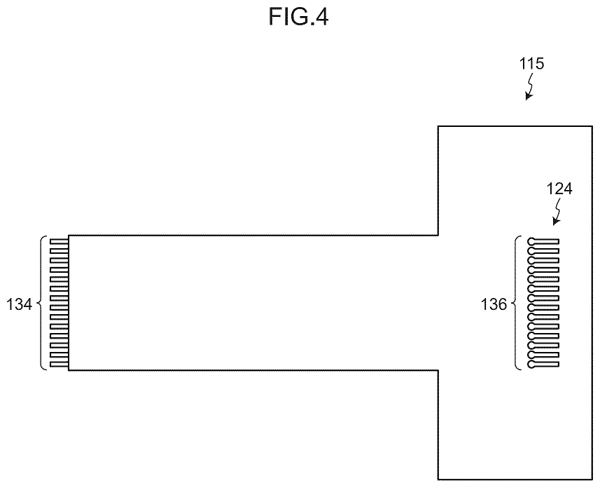

FIG. 3 is a top view of the FPC. FIG. 4 is a bottom view of the FPC. FIG. 3 and FIG. 4 illustrate states in which the FPC 115 that is folded as illustrated in FIG. 2 is planarly expanded. In FIG. 3 and FIG. 4, explanation will be given based on the assumption that an upper side corresponds to the proximal end side and a lower side corresponds to the distal end side.

The FPC 115 includes a first flexible printed circuit that is located on the proximal end side relative to a line B-B, and a second flexible printed circuit that is located on the distal end side relative to the first flexible printed circuit. The first flexible printed circuit and the second flexible printed circuit may be configured with a single flexible printed circuit, or each of the first flexible printed circuit and the second flexible printed circuit may be configured with a single flexible printed circuit. Meanwhile, in the first embodiment, the line B-B that separates the first flexible printed circuit and the second flexible printed circuit is set at a position at which the number of the ultrasonic transducers 111 included in the ultrasonic probe 10 is divided in half, but the position of the line B-B may be set arbitrarily. For example, it may be possible to set the position of the line B-B on the distal end side by setting the number of the ultrasonic transducers 111 connected to the first flexible printed circuit to double the number of the ultrasonic transducers 111 connected to the second flexible printed circuit, or it may be possible to randomly set the position of the line B-B.

As illustrated in FIG. 3, on the first surface of the FPC 115, wiring groups 121 to 123, connecting terminal groups 131 to 135, and an electrode portion 141 are formed. As illustrated in FIG. 4, on the second surface of the FPC 115, a wiring group 124 and a connecting terminal group 136 are formed. It is preferable that the wiring groups 121 to 124 are arranged regularly so as to extend linearly as illustrated in the drawings; however, the wiring groups 121 to 124 may extend randomly or arranged irregularly as a measure against noise or the like.

The wiring group 121 (first wiring group) extends from the ultrasonic transducers 111 toward the proximal end side, and electrically connects the ultrasonic transducers 111 and the signal lines 1181. The connecting terminal group 131 that is formed on a distal end of the wiring group 121 electrically connects the wiring group 121 and the ultrasonic transducers 111. The connecting terminal group 133 (first connecting terminal group) that is formed on a proximal end of the wiring group 121 electrically connects the wiring group 121 and the signal lines 1181.

The wiring group 122 (second wiring group) extends from the signal lines 1181 toward the distal end side. The connecting terminal group 135 (second connecting terminal group) that is formed on a proximal end of the wiring group 122 electrically connects the wiring group 122 and the signal lines 1181.

The wiring group 123 (third wiring group) extends from the ultrasonic transducers 111 toward the distal end side. The connecting terminal group 132 that is formed on a proximal end of the wiring group 123 electrically connects the wiring group 123 and the ultrasonic transducers 111. On a distal end of the wiring group 123, for example, the connecting terminal group 134 as a flying lead is formed.

The wiring group 124 (fourth wiring group) is connected to a via that is connected to a distal end of the wiring group 122 and that electrically connects the first surface and the second surface, and extends from the via toward the distal end side on the second surface. Specifically, the same number of wiring groups 124 as the wiring groups 122 are formed at positions facing the wiring groups 122 across the FPC 115. The connecting terminal group 136 that is formed on a distal end of the wiring group 124 electrically connects the wiring group 124 and the wiring group 123.

The electrode portion 141 is arranged in a region in which the ultrasonic transducers 111 are laminated, and arranged to prevent a difference in conditions for transmitting and receiving ultrasonic waves among the ultrasonic transducers 111.

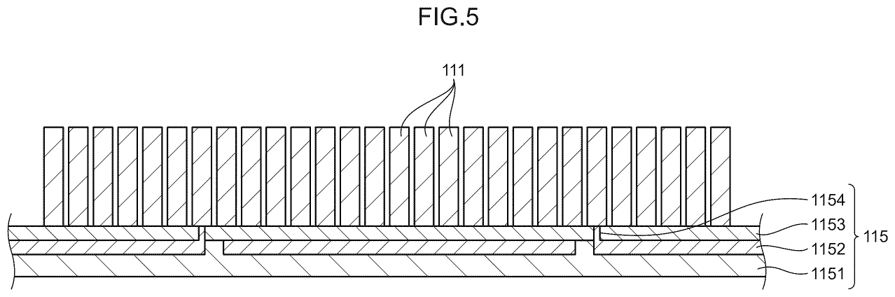

FIG. 5 is a partial cross-sectional view of the FPC. The FPC 115 includes a resin layer 1151, a conductive layer 1152 in which the wiring groups 121 to 123 are formed, an insulating layer 1153, and a via 1154 that is electrically connected to the ultrasonic transducers 111.

### Configuration of Connection Portion

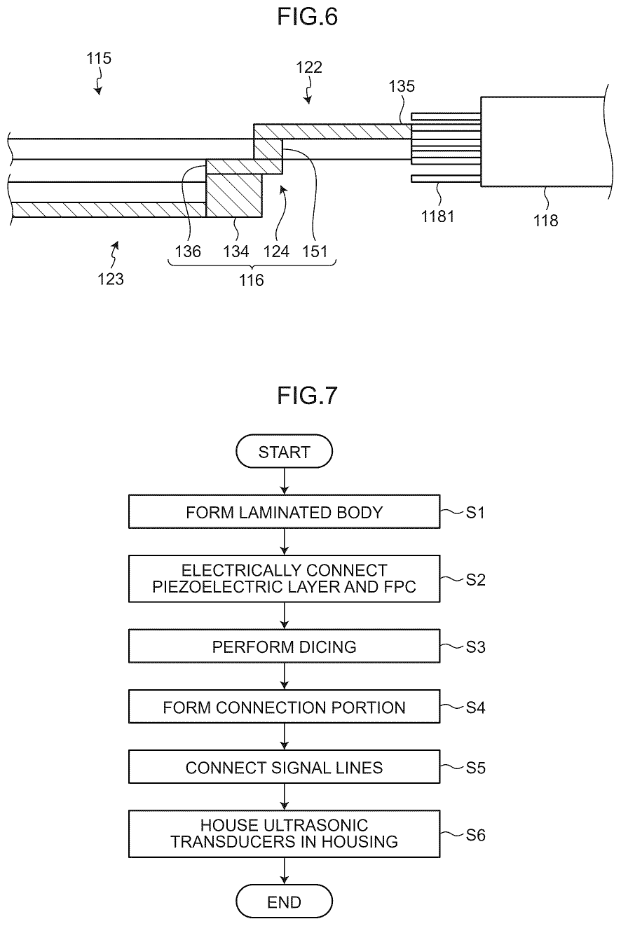

FIG. 6 is an enlarged view of the connection portion illustrated in FIG. 2. In FIG. 6, a right side is explained as the proximal end side and a left side is explained as the distal end side. The distal end side of the FPC 115 is folded to the proximal end side as illustrated in FIG. 2, and therefore, the connecting terminal group 134 that is formed at the distal end of the FPC 115 in FIG. 3 is located on the proximal end side of the wiring group 123 in FIG. 6.

As illustrated in FIG. 6, the connection portion 116 includes a via 151, the wiring group 124 (fourth wiring group), the connecting terminal group 136, and the connecting terminal group 134, and therefore electrically connects the wiring group 122 and the wiring group 123.

The via 151 electrically connects the first surface and the second surface. Specifically, the via 151 electrically connects the wiring group 122 and the wiring group 124.

The connecting terminal group 134 that is formed at the distal end of the wiring group 123 electrically connects the wiring group 123 and the wiring group 124.

### Method of Manufacturing Ultrasonic Probe

FIG. 7 is a flowchart illustrating a method of manufacturing the ultrasonic probe. First, piezoelectric layers that serve as the plurality of ultrasonic transducers 111 a laminated and a laminated body is formed (Step S1).

Subsequently, the piezoelectric layer as the laminated body and the FPC 115 are electrically connected (Step S2).

Thereafter, dicing for cutting the laminated body formed at Step S2 by dicing saucer is performed (Step S3). Accordingly, the piezoelectric element is cut and the plurality of ultrasonic transducers 111 in the form of cuboid are formed.

Further, the distal end side and the proximal end side of the FPC 115 are electrically connected to form the connection portion 116 (Step S4). In addition, the connection can be stabilized by connecting the FPC115 flat. Specifically, the connecting terminal group 136 and the connecting terminal group 134 of the FPC 115 are electrically connected and the distal end side of the FPC 115 and the proximal end side of the FPC 115 are fixed, so that the connection portion 116 is formed.

Further, the signal lines 1181 are electrically connected to the connecting terminal group 133 and the connecting terminal group 135 of the FPC 115 (Step S5). In this case, the distal end side and the proximal end side of the FPC 115 are fixed to each other by the connection portion 116, and therefore, it is possible to connect the signal lines 1181 and the FPC 115 by planarly expanding a portion in which the connecting terminal group 133 and the connecting terminal group 135 of the FPC 115 are formed. The connecting terminal group 133 and the connecting terminal group 135 are collectively formed on the same surface (first surface) of the FPC 115 on the proximal end side as illustrated in FIG. 3, and therefore, it is possible to ensure good operability and it is not needed to elongate the exposed portions 1181a.

Thereafter, the ultrasonic transducers 111 are housed in an opening of the housing 119 (Step S6). In this case, because the exposed portions 1181a are not long, so that, as illustrated in FIG. 2, it is possible to house the exposed portions 1181a inside the housing 119.

According to the first embodiment as described above, the FPC 115 electrically connects the ultrasonic transducers 111 and the signal lines 1181 and a relay board is not needed, so that it is possible to reduce a size of the distal end portion of the ultrasonic probe 10. Further, the connection portion 116 electrically connects the distal end side and the proximal end side of the FPC 115, and the connecting terminal group 133 and the connecting terminal group 135 that are connected to the signal lines 1181 are collectively formed on the same surface (first surface) of the FPC 115 on the proximal end side. As a result, it is possible to ensure good operability, it is not needed to elongate the exposed portions 1181a, and it is possible to house the exposed portions 1181a in the housing 119, so that it is possible to prevent reduction of flex resistance.

### First Modification

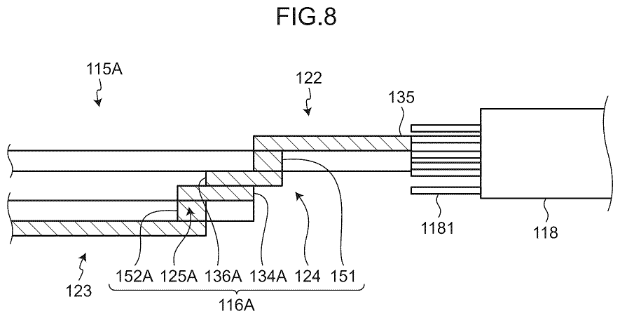

FIG. 8 is a partial enlarged view of a connection portion of an ultrasonic probe according to a first modification, and corresponds to an enlarged view of the connection portion illustrated in FIG. 2. As illustrated in FIG. 8, a connection portion 116A includes the via 151, the wiring group 124, a connecting terminal group 136A, a connecting terminal group 134A, a wiring group 125A, and a via 152A, and therefore electrically connects the wiring group 122 and the wiring group 123.

The connecting terminal group 136A that is formed at the distal end of the wiring group 124 electrically connects the wiring group 124 and the wiring group 125A.

The connecting terminal group 134A that is formed on a proximal end of the wiring group 125A (fifth wiring group) electrically connects the wiring group 124 and the wiring group 125A.

The connecting terminal group 136A and the connecting terminal group 134A include anisotropic conductive materials (not illustrated) and are electrically connected by, for example, an Anisotropic Conductive Film (ACF) or an Anisotropic Conductive Paste (ACP).

The wiring group 125A extends from the connecting terminal group 134A toward the distal end side.

The via 152A electrically connects the first surface and the second surface of an FPC 115A on the distal end side. Specifically, the via 152A electrically connects the wiring group 123 and the wiring group 125A.

As in the first modification as described above, the connection portion 116A may be an ACF or an ACP. Even in the first modification, with use of the FPC 115A, a relay board is not needed, so that it is possible to reduce the size of the distal end portion of the ultrasonic probe 10. In addition, the connecting terminal group 133 and the connecting terminal group 135 that are connected to the signal lines 1181 by the connection portion 116A are collectively formed on the same surface (first surface) of the FPC 115A on the proximal end side, so that it is possible to ensure good operability and prevent reduction of flex resistance.

Meanwhile, the connection portion is not specifically limited as long as it is possible to electrically connect the wiring group 122 and the wiring group 123. The connection portion may make physical contact of the connecting terminal and fix the connecting terminal by bonding or a tape, may mechanically crimp the connecting terminal, or may adopt welding, wire bonding, bonding using conductive resin, connection with a micro connector, non-contact power supply, or the like.

### Second Modification

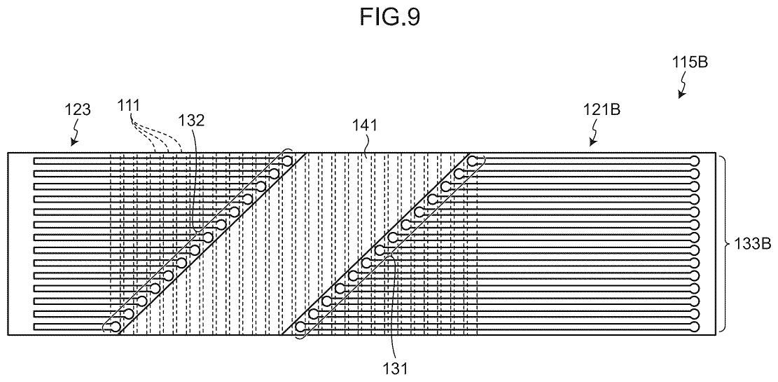

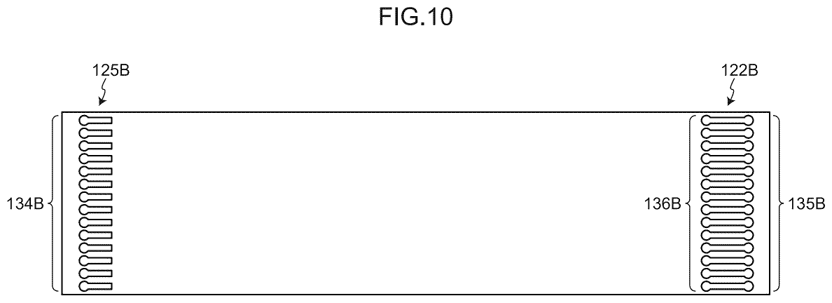

FIG. 9 is a top view of an FPC of an ultrasonic probe according to a second modification. FIG. 10 is a bottom view of the FPC of the ultrasonic probe according to the second modification. FIG. 9 and FIG. 10 are, similarly to FIG. 3 and FIG. 4, diagrams of a state in which the FPC that is folded as illustrated in FIG. 2 is planarly expanded. In FIG. 9 and FIG. 10, explanation will be given based on the assumption that an upper side corresponds to the proximal end side and a lower side corresponds to the distal end side.

As illustrated in FIG. 9, on the first surface of an FPC 115B, a wiring group 121B, the wiring group 123, the connecting terminal groups 131 and 132, a connecting terminal group 133B, and the electrode portion 141 are formed. On the second surface of the FPC 115B, as illustrated in FIG. 10, wiring groups 122B and 125B and connecting terminal groups 134B, 135B, and 136B are formed. In this configuration, the wiring group 121B is formed on the first surface side and the wiring group 122B is formed on the second surface side, so that it is not needed to increase a width of the FPC 115 at the proximal end side as in FIG. 3 and FIG. 4.

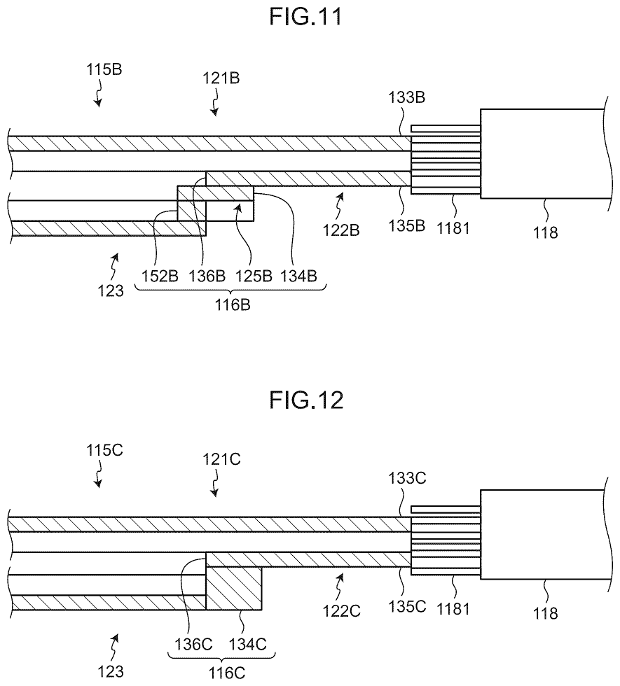

FIG. 11 is a partial enlarged view of a connection portion of the ultrasonic probe according to a second modification, and corresponds to an enlarged view of the connection portion illustrated in FIG. 2. As illustrated in FIG. 11, a connection portion 116B includes the connecting terminal group 136B, the connecting terminal group 134B, the wiring group 125B (fifth wiring group), and a via 152B, and therefore electrically connects the wiring group 122B and the wiring group 123.

The wiring group 121B (first wiring group) is formed on the first surface, extends from the ultrasonic transducers 111 toward the proximal end side, and electrically connects the ultrasonic transducers 111 and the signal lines 1181. The connecting terminal group 133B (first connecting terminal group) that is formed on a proximal end of the wiring group 121B electrically connects the wiring group 121B and the signal lines 1181.

The wiring group 122B (second wiring group) is formed on the second surface, and extends from the signal line 1181 toward the distal end side. The connecting terminal group 135B (second connecting terminal group) that is formed on a proximal end of the wiring group 122B electrically connects the wiring group 122B and the signal line 1181.

The connecting terminal group 136B that is formed on a distal end of the wiring group 122B electrically connects the wiring group 122B and the wiring group 125B.

The connecting terminal group 134B that is formed on a proximal end of the wiring group 125B electrically connects the wiring group 122B and the wiring group 125B. Here, by arranging the wiring group 121C on the first side and the wiring group 122C on the second side so that they do not overlap each other, the heat of the solder connection on the first side is transmitted to the solder connection on the second side, and the solder on the second side can be prevented from melting.

The wiring group 125B extends from the connecting terminal group 136B toward the distal end side.

The via 152B electrically connects the first surface and the second surface of the FPC 115B on the distal end side. Specifically, the via 152B electrically connects the wiring group 123 and the wiring group 125B.

According to the second modification as described above, the wiring group 122B may be formed on the second surface. Even in the second modification, with use of the FPC 115B, a relay board is not needed, so that it is possible to reduce the size of the distal end portion of the ultrasonic probe 10. In addition, the connecting terminal group 133B and the connecting terminal group 135B that are connected to the signal lines 1181 by the connection portion 116B are collectively formed on the proximal end side of the FPC 115B, so that it is possible to improve operability and prevent reduction of flex resistance.

### Third Modification

FIG. 12 is a partial enlarged view of a connection portion of an ultrasonic probe according to a third modification, and corresponds to an enlarged view of the connection portion illustrated in FIG. 2. As illustrated in FIG. 12, a connection portion 116C includes a connecting terminal group 136C and a connecting terminal group 134C, and therefore electrically connects a wiring group 122C and the wiring group 123.

A wiring group 121C (first wiring group) is formed on the first surface, extends from the ultrasonic transducers 111 toward the proximal end side, and electrically connects the ultrasonic transducers 111 and the signal lines 1181. A connecting terminal group 133C (first connecting terminal group) that is formed on a proximal end of the wiring group 121C electrically connects the wiring group 121C and the signal lines 1181.

The wiring group 122C (second wiring group) is formed on the second surface, and extends from the signal lines 1181 toward the distal end side. A connecting terminal group 135C (second connecting terminal group) that is formed on a proximal end of the wiring group 122C electrically connects the wiring group 122C and the signal lines 1181.

In this configuration, similarly to the second modification, the wiring group 121C is formed on the side of the first surface and the wiring group 122C is formed on the side of the second surface, so that it is not needed to increase the width of the FPC 115 at the proximal end side as illustrated in FIG. 3.

The connecting terminal group 136C that is formed on a distal end of the wiring group 122C electrically connects the wiring group 122C and the wiring group 123.

The connecting terminal group 134C that is formed at the proximal end of the wiring group 123 electrically connects the wiring group 122C and the wiring group 123.

As in the third modification as described above, the wiring group 122C may be formed on the second surface. Even in the third modification, with use of an FPC 115C, a relay board is not needed, so that it is possible to reduce the size of the distal end portion of the ultrasonic probe 10. In addition, the connecting terminal group 133C and the connecting terminal group 135C that are connected to the signal line 1181 by the connection portion 116C are collectively formed on the proximal end side of the FPC 115C, so that it is possible to ensure good operability and prevent reduction of flex resistance.

### Fourth Modification

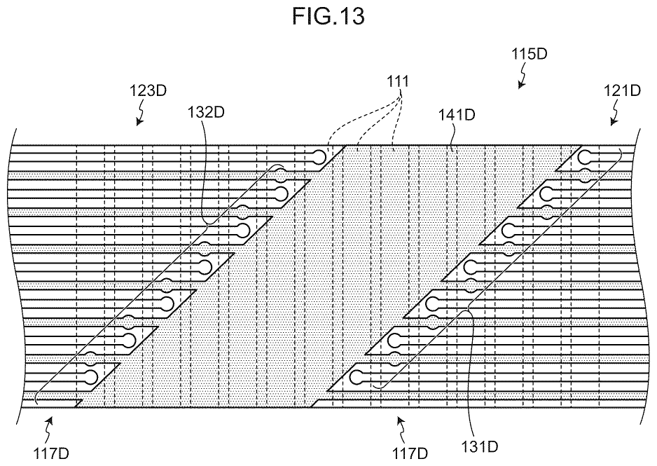

FIG. 13 is a partial enlarged view of an FPC of an ultrasonic probe according to a fourth modification. As illustrated in FIG. 13, an FPC 115D includes wiring groups 121D and 123D, connecting terminal groups 131D and 132D, an electrode portion 141D, and a ground wiring 117D. the wiring group 121D or 123D and the ground wiring 117D are alternately arranged in a direction crossing an extending direction of each wiring.

The wiring group 121D (first wiring group) extends from the ultrasonic transducers 111 toward the proximal end side, and electrically connects the ultrasonic transducers 111 and the signal lines 1181. The connecting terminal group 131D that is formed on a distal end of the wiring group 121D electrically connects the wiring group 121D and the ultrasonic transducers 111.

The wiring group 123D (third wiring group) extends from the ultrasonic transducers 111 toward the distal end side. The connecting terminal group 132D that is formed at the proximal end of the wiring group 123D electrically connects the wiring group 123D and the ultrasonic transducers 111.

The electrode portion 141D is arranged in a region in which the ultrasonic transducers 111 are laminated, and is arranged to prevent a difference in conditions for transmitting and receiving ultrasonic waves among the ultrasonic transducers 111. The electrode portion 141D is electrically connected to the ground wiring 117D.

A proximal end side of the ground wiring 117D is electrically connected to the ground line of the signal cable 118. In this case, the ground line 117 illustrated in FIG. 2 is not needed.

As in the fourth modification as described above, it may be possible to form the ground wiring 117D in the FPC 115D.

### Second Embodiment

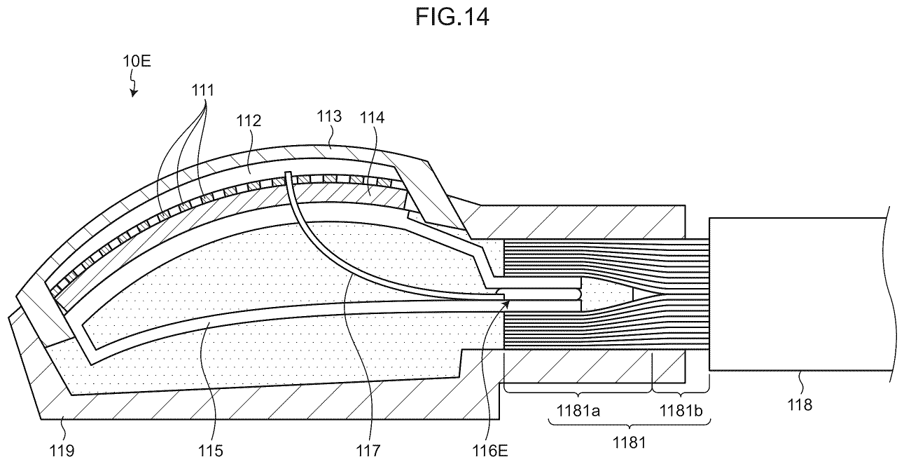

FIG. 14 is a cross-sectional view of an ultrasonic probe according to a second embodiment. As illustrated in FIG. 14, an ultrasonic probe 10E includes a bonding portion 116E that bonds a portion of the second surface of the FPC 115 at the proximal end side and a portion of the second surface of the FPC 115 at the distal end side.

The bonding portion 116E bonds the portion of the FPC 115 at the proximal end and the portion of the FPC 115 at the distal end side, but does not include an electrical connection portion. Therefore, the wiring group 121 that extends from the ultrasonic transducers 111 to the proximal end side is electrically connected to the exposed portions 1181a without any change. Similarly, the wiring group 123 that extends from the ultrasonic transducers 111 to the distal end side is electrically connected to the exposed portions 1181a without any change.

According to the second embodiment as described above, the FPC 115 electrically connects the ultrasonic transducers 111 and the signal lines 1181, so that a relay board is not needed and it is possible to reduce a size of a distal end portion of the ultrasonic probe 10E. Further, it is possible to connect the exposed portions 1181a while the bonding portion 116E bonds the portion of the FPC 115 at the proximal end and the portion of the FPC 115 at the distal end side, so that it is possible to ensure good operability, it is not needed to increase lengths of the exposed portions 1181a, it is possible to house the exposed portions 1181a in the housing 119, and it is possible to prevent reduction of flex resistance.

### Note 1

A method of manufacturing an ultrasonic probe that includes
a plurality of ultrasonic transducers that are arranged along an arrangement direction from a first side toward a second side;
a first flexible printed circuit including
   a first wiring group that extends from the plurality of ultrasonic transducers toward the first side and electrically connects the plurality of ultrasonic transducers and a plurality of signal lines configured to transmit and receive ultrasonic signals to and from the plurality of ultrasonic transducers; and
   a second wiring group that extends from the plurality of signal lines toward the second side; and
a second flexible printed circuit including a third wiring group that extends from the plurality of ultrasonic transducers toward the second side,
the method comprising:
electrically connecting the second wiring group and the third wiring group; and
electrically connecting the plurality of signal lines to the first wiring group and the second wiring group.

### Note 2

A method of manufacturing an ultrasonic probe that includes
a plurality of ultrasonic transducers that are arranged along an arrangement direction from a first side toward a second side;
a signal cable including a plurality of signal lines, each signal line being electrically connected to a corresponding one of the plurality of ultrasonic transducers to transmit and receive an ultrasonic signal to and from the corresponding one of the plurality of ultrasonic transducers, each signal line
   an exposed portion which is located on a distal end of the single cable and at which a conducting wire for transmitting and receiving the ultrasonic signal is exposed, and
   a covered portion which is located on a proximal end side of the exposed portion and at which the conducting wire is covered by an outer skin; and
a flexible printed circuit including
   a first wiring group that extends from the plurality of ultrasonic transducers toward the first side and electrically connects the plurality of ultrasonic transducers and the plurality of signal lines; and
   a third wiring group that extends from the plurality of ultrasonic transducers toward the second side and electrically connects the plurality of ultrasonic transducers and the plurality of signal lines,
the method comprising:
bonding a portion of the flexible printed circuit at the first side and a portion of the flexible printed circuit at the second side;
electrically connecting the plurality of signal lines to the first wiring group and the third wiring group; and
housing the plurality of ultrasonic transducers, the flexible printed circuit, and exposed portions of the plurality of signal lines in a housing.

According to the disclosure, it is possible to realize an ultrasonic probe and an ultrasonic endoscope capable of reducing a size of a distal end portion and prevent reduction of flex resistance.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic probe comprising:
a plurality of ultrasonic transducers that are arranged along an arrangement direction from a first side toward a second side;
a first flexible printed circuit including
a first wiring group that extends from the plurality of ultrasonic transducers toward the first side and electrically connects the plurality of ultrasonic transducers and a plurality of signal lines configured to transmit and receive ultrasonic signals to and from the plurality of ultrasonic transducers; and
a second wiring group that extends from the plurality of signal lines toward the second side;
a second flexible printed circuit including a third wiring group that extends from the plurality of ultrasonic transducers toward the second side; and
a connection portion that electrically connects the second wiring group and the third wiring group.

2. The ultrasonic probe according to claim 1, further comprising:
a signal cable that includes the plurality of signal lines that are electrically connected to the plurality of ultrasonic transducers.

3. The ultrasonic probe according to claim 2, wherein the plurality of ultrasonic transducers are located on the second side relative to the signal cable.

4. The ultrasonic probe according to claim 1, wherein the connection portion is located on the first side relative to the plurality of ultrasonic transducers.

5. The ultrasonic probe according to claim 1, wherein the first flexible printed circuit and the second flexible printed circuit are configured with a single flexible printed circuit.

6. The ultrasonic probe according to claim 2, wherein
the first flexible printed circuit includes
a first connecting terminal group that electrically connects the first wiring group and the plurality of signal lines, and
a second connecting terminal group that electrically connects the second wiring group and the plurality of signal lines.

7. The ultrasonic probe according to claim 6, wherein the first connecting terminal group and the second connecting terminal group are formed on a first surface.

8. The ultrasonic probe according to claim 6, wherein
the first connecting terminal group is formed on a first surface, and
the second connecting terminal group is formed on a second surface that is a different surface from the first surface.

9. The ultrasonic probe according to claim 8, wherein
the first connecting terminal group on the first surface and the second connecting terminal formed on the second surface are not overlapped each other.

10. The ultrasonic probe according to claim 1, wherein the connection portion is configured to fix the first flexible printed circuit and the second flexible printed circuit.

11. The ultrasonic probe according to claim 10, wherein the first flexible printed circuit and the second flexible printed circuit are flat each other at the connection portion.

12. The ultrasonic probe according to claim 7, wherein
the connection portion includes
a via that is electrically connected to the second wiring group and electrically connects the first surface and a second surface that is a different surface from the first surface, and
a fourth wiring group that extends from the via toward the second side and electrically connects the via and the third wiring group.

13. The ultrasonic probe according to claim 12, wherein the connection portion includes an anisotropic conductive material.

14. The ultrasonic probe according to claim 12, wherein a flying lead is formed at a distal end of the third wiring group.

15. An ultrasonic probe comprising:
a plurality of ultrasonic transducers that are arranged along an arrangement direction from a first side toward a second side;
a signal cable including a plurality of signal lines, each signal line being electrically connected to a corresponding one of the plurality of ultrasonic transducers to transmit and receive an ultrasonic signal to and from the corresponding one of the plurality of ultrasonic transducers, each signal line including
an exposed portion which is located at a distal end of the single cable and at which a conducting wire for transmitting and receiving the ultrasonic signal is exposed, and
a covered portion which is located on a proximal end side of the exposed portion and at which the conducting wire is covered by an outer skin;
a flexible printed circuit including
a first wiring group that extends from the plurality of ultrasonic transducers toward the first side and electrically connects the plurality of ultrasonic transducers and the plurality of signal lines; and
a third wiring group that extends from the plurality of ultrasonic transducers toward the second side and electrically connects the plurality of ultrasonic transducers and the plurality of signal lines;
a housing that houses the plurality of ultrasonic transducers, the flexible printed circuit, and exposed portions of the plurality of signal lines; and
a bonding portion that bonds a portion of the flexible printed circuit at the first side and a portion of the flexible printed circuit at the second side.

16. The ultrasonic probe according to claim 15, wherein, on the flexible printed circuit, a second wiring group that extends from the plurality of signal lines toward the second side and electrically connects the third wiring group and the plurality of signal lines is formed.

17. The ultrasonic probe according to claim 15, wherein
the first wiring group and the third wiring group are formed on a first surface of the flexible printed circuit, and the bonding portion bonds a portion of a second surface of the flexible printed circuit at the first side and a portion of the second surface of the flexible printed circuit at the second side, the second surface being a different surface from the first surface.

18. An ultrasonic endoscope comprising:
an ultrasonic probe that includes
a plurality of ultrasonic transducers that are arranged along an arrangement direction from a first side toward a second side;
a first flexible printed circuit including
a first wiring group that extends from the plurality of ultrasonic transducers toward the first side and electrically connects the plurality of ultrasonic transducers and a plurality of signal lines configured to transmit and receive ultrasonic signals to and from the plurality of ultrasonic transducers; and
a second wiring group that extends from the plurality of signal lines toward the second side;
a second flexible printed circuit including a third wiring group that extends from the plurality of ultrasonic transducers toward the second side;
a connection portion that electrically connects the second wiring group and the third wiring group; and
a housing that houses the plurality of ultrasonic transducers;
a rigid material that is connected to the first side of the ultrasonic probe;
a bending portion that is connected to the rigid material at the first side and that is bendable;
a flexible tube that is connected to the bending portion at the first side and that has flexibility;
an insertion portion configured to be inserted into a subject; and
an operating portion that is arranged on the first side relative to the insertion portion and is configured to receive operation of bending the bending portion, wherein the connection portion is located on the second side relative to the bending portion.
